(19) United States
(12) Patent Application Publication
(10) Pub. No.: US 2024/0269007 A1
(43) Pub. Date: Aug. 15, 2024

(54) BANDAGE FOR ENCLOSING A LEG REGION OR ARM REGION OF A LIVING BEING

(71) Applicant: KOB GmbH, Wolfstein (DE)

(72) Inventor: SEBASTIAN CAMERER, Bolzano (IT)

(21) Appl. No.: 18/569,393

(22) PCT Filed: Jun. 13, 2022
(86) PCT No.: PCT/EP2022/065979

§ 371 Date: Dec. 12, 2023

(30) Foreign Application Priority Data
Jun. 14, 2021 (DE) 10 2021 115 370.0

(51) Int. Cl. A61F 13/10; A61F 13/00; A61F 13/06

(52) U.S. Cl. CPC A61F 13/10; A61F 13/06; A61F 2013/00093; A61F 2013/00165; A61F 2013/0028

(57) ABSTRACT

The invention relates to a bandage (100) for enclosing a leg region or arm region of a living being, said bandage comprising: an elongate, tubular bandage part (10), at least part of which has a single slit in the longitudinal direction (LR) and which is designed to elastically enclose the leg or arm region when worn; and a closure part (20) which has closure elements (22) and which is fixedly connected to one end of the slit region (B) of the bandage part in order to detachably seal the slit region (B) by connecting the adjacent ends of the slit region (B) in an overlapping manner by means of the closure elements (22, 22′). According to the invention, the bandage part (10) has different compression zones (K1, K2) in the longitudinal direction (LR) which each have different radial compression strengths transverse to the longitudinal direction (L).

The invention relates to a bandage, in particular a ready-to-use secondary bandage, for enclosing a leg region or arm region of a living being.

The use of secondary bandages for fixing and protecting primary wound dressings in medical treatments such as, for example, wound treatments, is known. Bandages of this type are used in stationary care such as, for example, hospitals, care homes or other institutions, and in outpatient care, for example homecare of elderly patients. Here, products such as, for example, wrap bandages, tubular bandages and padded bandages are often used, which in some instances have to be combined with one another in multiple layers and require trained, skilled expertise, thus excluding the use by laypersons. By way of example, such care is the use of a wrap bandage for the treatment of wounds on body parts such as legs or arms. During application, slippage of the primary wound dressing and the creation of creases has to be avoided so as to preclude any bruises. Furthermore, at the same time the bandage has to be applied in a sufficiently tight and uniform manner on the body part, without exerting dangerous overcompression in the process. Subsequent readjusting is impeded in that this requires the bandage to be re-wrapped or completely cut open. The primary wound dressing can slip or be damaged in the process, which overall results in an increased effort in terms of time and costs.

Consequently, it is an object of the present invention to provide a ready-to-use bandage, in particular a ready-to-use secondary bandage, which combines the advantages of a padded bandage, a tubular bandage and an elastic bandage in one product, offers easy handling without specially trained skills of the art being required for applying the bandage, and which at the same time provides a better fit and avoids creasing.

This object is achieved by a bandage as claimed in patent claim 1, Advantageous design embodiments and refinements of the invention are described in the dependent claims.

This object is achieved in particular by a bandage for enclosing a leg region or arm region of a living being, having: an elongate, tubular bandage part which is at least partially slit once in the longitudinal direction and which in an applied state is adapted to elastically enclose the arm or leg region; and a fastener part which has fastener elements and is fixedly connected to one end of the slit region of the bandage part so as to releasably close the slit region by way of an overlapping connection of the adjacent ends of the slit region by means of the fastener elements. It is provided herein that the bandage part in the longitudinal direction has dissimilar compression zones having in each case dissimilar radial compression strengths transverse to the longitudinal direction or in each case dissimilar elasticity moduli of the bandage material.

Provided in this way is an adaptable ready-to-use calf bandage, lower leg bandage or lower arm bandage which covers the calf region or the lower leg region from the ankle to the knee, or the lower arm from the elbow to the hand.

A positive adaptation of the bandage to a leg region or arm region by means of the dissimilar compression zones is implemented herein without specialist skills being required when applying the bandage.

The dissimilar compression zones herein can be assigned to predetermined regions of a leg, in particular of a lower leg, or of an arm, in particular of a lower arm.

In this context, a first compression zone in the bandage part can be assigned to a collar region on the knee and/or on the ankle of the lower leg, and a second compression zone in the bandage part can be assigned to a calf region of the lower leg. The first compression zone herein can have a higher compression strength than the second compression zone. As a result, potential slipping of the bandage is further reduced because the bandage bears on the knee and/or on the ankle by way of a higher compression. At the same time, it can be prevented by the second compression zone that the bandage bears too firmly on a wound, for example, or exerts too much pressure on the latter.

The bandage part can be produced from at least one blank of a flat material which comprises a non-woven material.

In a further embodiment, the bandage part can be produced from at least one blank of a stretch material.

The stretch material herein can have an elasticity of at least 50% and a resilience of 30% to 100%.

In one embodiment, the stretch material can have a 2-way stretch fabric, wherein one stretch direction runs in the transverse direction of the bandage.

In a further embodiment, the stretch material can have a 4-way stretch fabric, wherein one stretch direction runs in the transverse direction and a further stretch direction runs in the longitudinal direction of the bandage. Additional flexibility is provided as a result. Depending on the requirements set for the bandage, either the 2-way stretch fabric or the 4-way stretch fabric may be used.

The stretch material can furthermore comprise elastomer threads or elastane fibers of dissimilar thicknesses which generate dissimilar compression strengths in the respective dissimilar compression zones. Zones in which the thicker thread diameters result in a higher compression strength than zones with thinner thread diameters can be formed by means of the elastomer threads of dissimilarly thick configurations.

Furthermore, the stretch material can comprise elastomer threads or elastane fibers with dissimilar preloads which generate dissimilar compression strengths in the respective dissimilar compression zones.

The bandage part next to the slit region can comprise a distal region which is closed in a tubular manner and when applying the bandage is pulled over a foot or a hand of the living being, and in the applied state of the bandage is adjacent to the foot or the hand. This can facilitate the handling of the bandage. For example, the closed distal region prevents that the bandage slides away over an ankle or wrist when being applied, so that the bandage can be easily and rapidly closed.

The bandage part herein, in an applied state, in its distal region which is closed in a tubular manner, can enclose in a compressing manner an ankle region of a foot, and in its slit region can enclose in a compressing manner a calf region of a lower leg.

The fastener part as fastener elements can comprise individual hook and loop fastener parts which are punched out of a hook and loop fastener tape and are individually connected to the bandage part. The hook and loop fastener parts enable simple and reliable fastening of the ends of the bandage part in the slit region.

In a further embodiment, the fastener part can also be configured as an integral multi-hook and loop fastener part which on the one side thereof, in its non-hook region, is connected to the bandage part, and on the opposite side thereof, in its hook region, has projecting fastener elements which are configured by punching the hook and loop fastener tape.

Furthermore, the bandage part can be produced from two blanks of identical shape, which are connected to one another by a seam which is elastic in a seam direction.

The bandage part can have a shape adapted to a calf region of a lower leg, as a result of which said bandage part is configured for an even more exact fit.

Furthermore, the bandage can be configured as a hygienic single-use product.

The invention will be explained in more detail hereunder by way of example by means of the drawings in which:

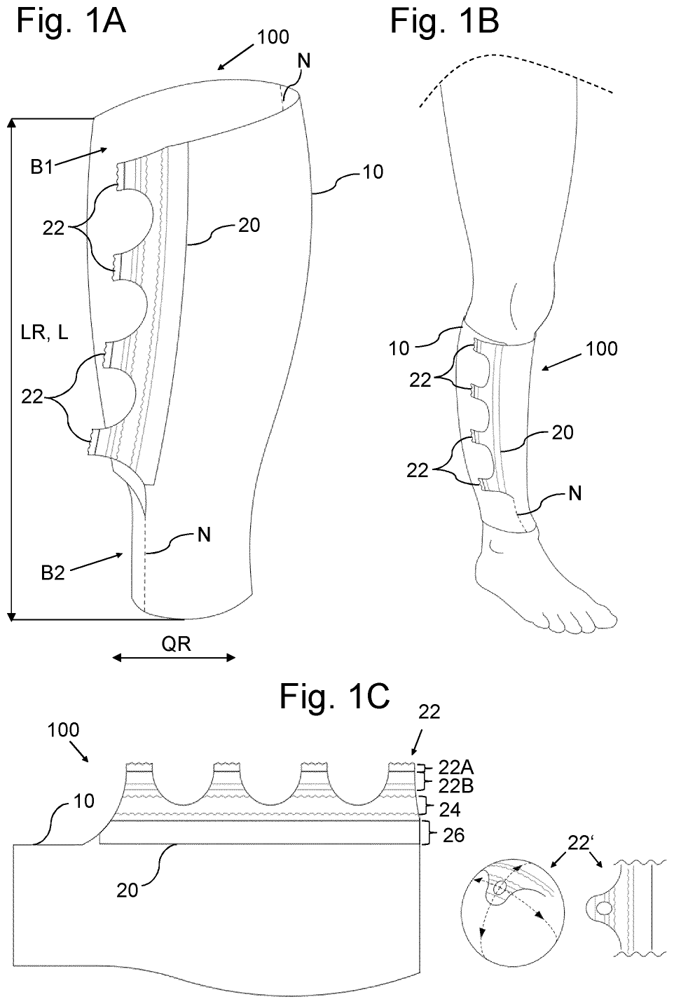

FIG. 1A shows a schematic perspective view of a bandage according to one exemplary embodiment of the invention;

FIG. 1B shows a schematic perspective view of the bandage according to one exemplary embodiment of the invention, in the closed state;

FIG. 1C shows a schematic lateral view of a bandage part of the bandage according to one exemplary embodiment of the invention;

FIG. 1D shows a schematic lateral view of the bandage part of the bandage according to one exemplary embodiment of the invention;

FIG. 1E shows a schematic exploded drawing of the bandage according to one exemplary embodiment of the invention;

FIG. 1F shows a schematic perspective view of the bandage according to a further exemplary embodiment of the invention, in the closed state;

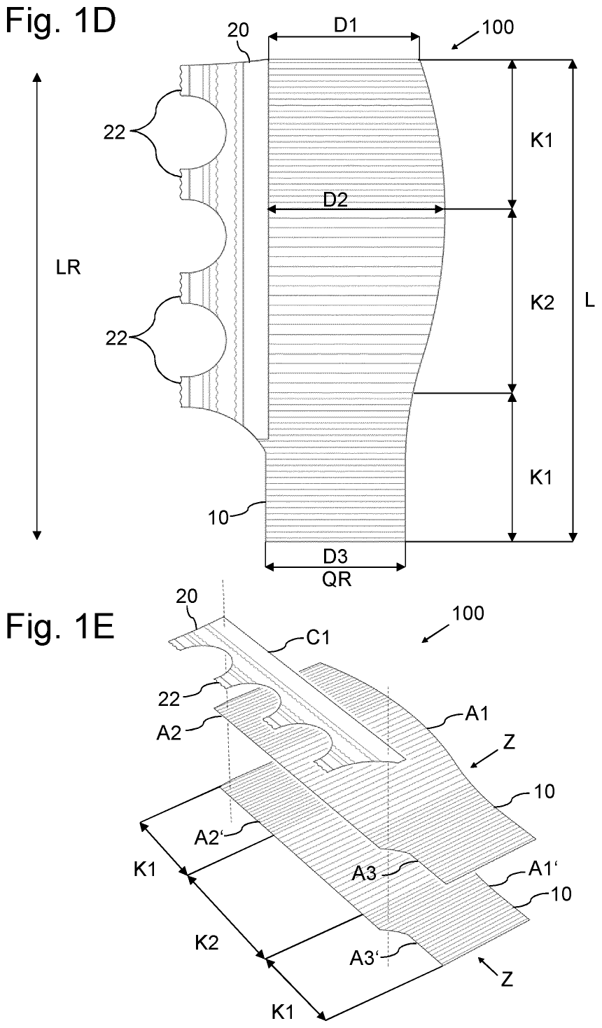

FIG. 2 shows a schematic lateral view of the bandage according to a plurality of exemplary embodiments of the invention;

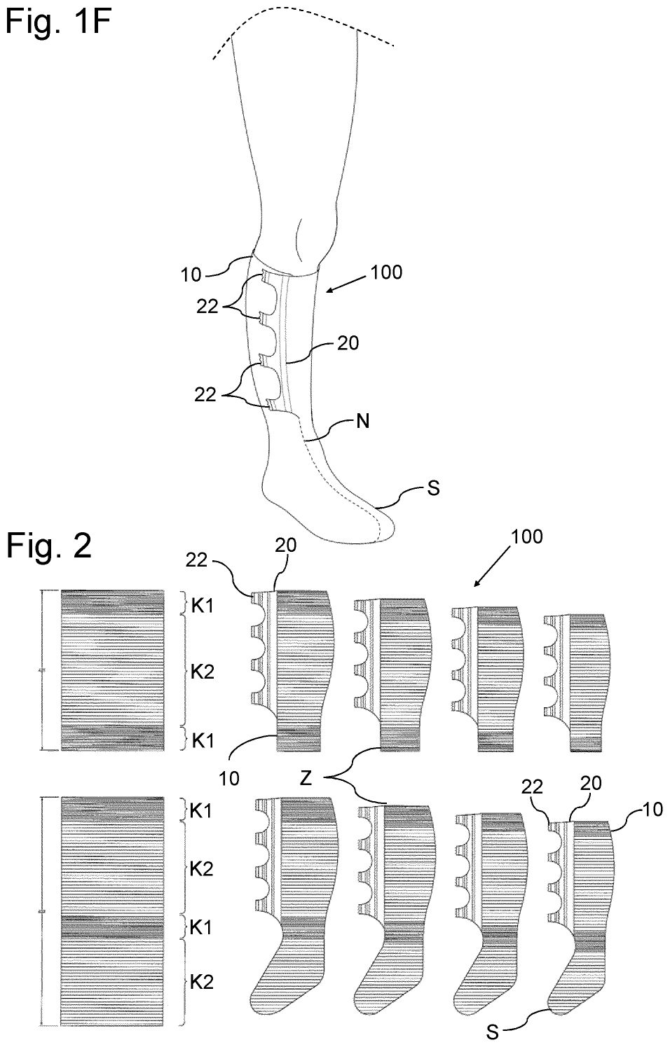

FIG. 3 shows schematic cutting patterns for producing the bandage according to a plurality of exemplary embodiments of the invention; and

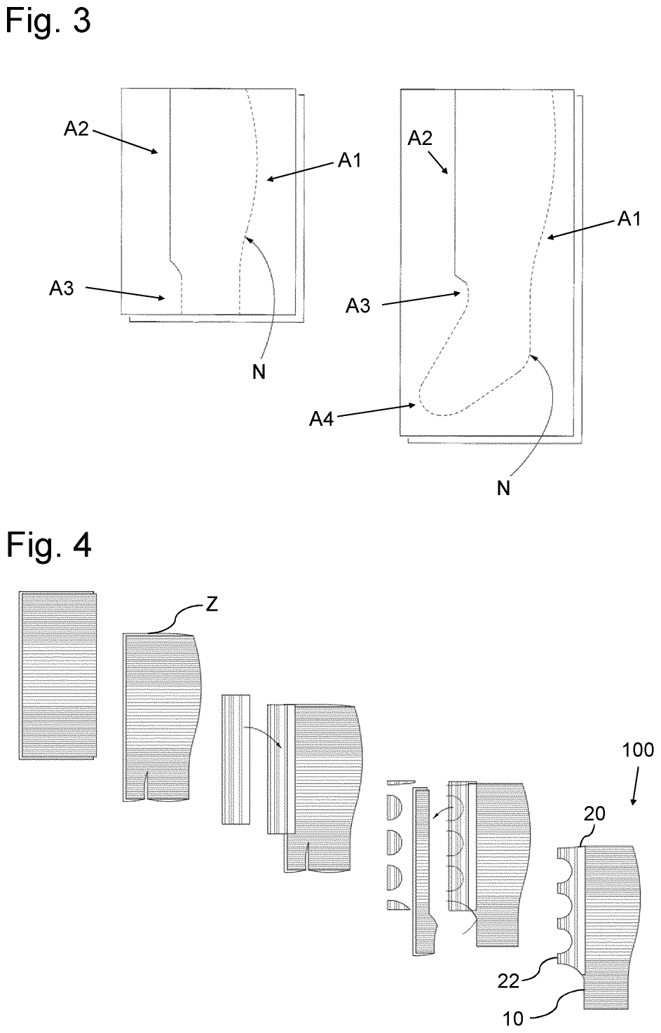

FIG. 4 shows a schematic illustration of a production of the bandage according to one exemplary embodiment of the invention.

The same reference signs are used hereunder for identical and equivalent components.

FIG. 1A shows a schematic perspective view of a bandage 100 for enclosing a leg region or arm region of a living being. The living being may be a human or else an animal. The bandage 100 comprises an elongate tubular bandage part 10 which is at least partially slit in the longitudinal direction LR. In FIG. 1A, this is indicated in a simplified manner by an arrow pointing to the slit region B1. In an applied state, the bandage part 10 elastically encloses the leg region or arm region. In this context, FIG. 1B shows the bandage 100 in the closed state, whereby a leg region is illustrated by way of example. However, an arm region is likewise possible.

The bandage 100 furthermore comprises a fastener part 20 having fastener elements 22, 22′. The fastener part 20 is fixedly connected to one end of the slit region B1 of the bandage part 10 so as to releasably close the slit region B1 by way of an overlapping connection of the adjacent ends of the slit region B1 by means of the fastener elements 22, 22′. This is illustrated in a simplified manner in FIG. 1A and FIG. 1B.

The length of the slit herein can vary, depending on the requirements set for the bandage 100. For example, said length can be larger than 10%, or larger than 20%, or larger than 30%, or larger than 40%, or larger than 50%, or larger than 60%, or larger than 70%, or larger than 80%, or larger than 90%, or larger than up to 100% of the overall length L of the bandage part 10. However, it is furthermore possible that the length of the slit is 100% of the overall length L, the length of the slit region B1 thus being equal to the length LR, i.e. the tubular bandage part 10 being completely slit or cut open in the longitudinal direction L.

The fastener part 20 can be formed by a plurality of elements 22A, 22B, 24,26, which is illustrated in FIG. 1C. FIG. 1C shows a schematic lateral view of the bandage part 10, whereby a fastener element 22 can be produced from a grip part 22A and a hook part 22B such as a hook and loop fastener part, for example. The grip part 22A allows a user to grip the fastener element 22. Shown in this context is a further embodiment of a fastener element 22′ on the right side in FIG. 1C, which is configured as a fastener tab with a hole or a clearance in the hook part 22B. The clearance herein can be of a circular or oval or elliptic shape. This can facilitate gripping and provide better control for a finger of the user. The hole herein functions as a gripping eyelet for the tip of the finger. This design embodiment enables a planar crease-free application on curved surfaces, which in turn can result in better adhesion and reduced creasing. Apart from the fastener tab with hole or oval clearance illustrated in FIG. 1C, further design embodiments are possible. The fastener part 20 can furthermore have an elastic part 24 and a connection part 26. The connection part 26 can, for example, connect the fastener part 20 to the bandage part 10 by means of adhesive bonding or welding.

The bandage part 10 in the longitudinal direction LR furthermore has dissimilar compression zones K1, K2 having respective different radial compression strengths transverse to the longitudinal direction LR (i.e. in the transverse direction QR).

As a result, an optimally adapted application of the bandage 100 on corresponding locations of the leg region or arm region is implemented. The compression zones K1, K2 enable simplified handling because no special prior knowledge is required for the application, and corresponding compression at the respective locations is predefined. Furthermore, creasing is further reduced as a result.

The dissimilar compression zones K1, K2 can be assigned to, for example, predetermined regions of a leg, in particular of a lower leg, or of an arm, in particular of a lower arm. FIG. 1D shows a schematic lateral view of the bandage part 10 in which the compression zones K1, K2 are illustrated. As can be seen from FIG. 1D in combination with FIGS. 1A and 1B, a first compression zone K1 in the bandage part 10 can be assigned to a collar region on the knee and/or on the ankle of the lower leg. Furthermore, a second compression zone K2 in the bandage part 10 can be assigned to a calf region of the lower leg. The first compression zone K1 herein can have a higher compression strength than the second compression zone K2, this being shown in a simplified manner by the horizontal lines in FIG. 1D, which are representative of corresponding material threads and the mutual spacing thereof. The elastic threads, elastane threads, elastane fibers or elastomer threads will be described in detail later with reference to FIG. 2.

The sizing of the bandage part 10 shown in FIG. 1D here may differ. For example, an upper diameter D1 can be in a range from 50 mm to 150 mm. A central diameter D2 can be in a range from 100 mm to 200 mm. And a lower diameter D3 can be in a range from 50 mm to 150 mm. The length L of the bandage part 10 herein can be in the range from 100 mm to 400 mm. A length ratio between a compression zone K1, K2 and the overall length L can be in a range between 10% to 70%, or between 10% to 50%, or between 20% to 40%, or between 25% and 35%.

FIG. 1E shows a schematic exploded drawing of the bandage 100, wherein it can be seen that the bandage part 10 can be formed from two blanks Z which are of identical shape and are to be connected to one another, and from the fastener part 20 described above. Both blanks Z comprise the dissimilar compression zones K1, K2, this being indicated in a simplified manner by arrows in in FIG. 1E. For example, the blanks Z can be connected to one another by ultrasonic welding at corresponding cut edge portions A1, A1′. The cut edge portions A1, A1′ of the respective blanks Z have an arcuate or curved shape, which in the example shown in FIG. 1E corresponds to a calf region or lower leg and results from a predefined cutting pattern. The cutting edge portions herein are curved so as to be S-shaped, having an upper convex curvature region with a curvature radius between 300 mm and 450 mm in the calf region, and a lower concave curvature region with a curvature radius between 250 mm and 350 mm, wherein the lower curvature region in this instance opens into a straight cuff portion in the ankle region. The cut edge portions A2, A2′ are rectilinear and are not connected to one another. They form the slit region B1, or an opening, respectively. However, the cut edge portion A1 in the upper blank Z is connected to a likewise rectilinear cut edge portion C1 of the fastener part 20. This can take place by means of the connection part 26 described above, for example. The cut edge portions A3, A3′ are likewise connected to one another and form a closed distal region B2 which is yet to be described in detail at a later stage. As can be seen from FIG. 1E, the cut edge portions A3, A3′ likewise have an arcuate or curved shape which follows the cutting pattern and in the connected state encloses an ankle. It is likewise possible for the bandage part 10 to be integrally configured. The production of the bandage 100 will be explained in detail later with reference to FIGS. 3 and 4. The ratio between the lengths of the slit region B1 and the closed region B2 herein can be in a range between 5% and 40%, or between 10% and 30%, or between 15% and 25%. In these cases, the closed region B2 can be easily pulled over the foot or the hand, while at least more than half of the bandage 100 is able to be closed with variable compression by means of the fastener part 20. Purely theoretically, the bandage part 10 in the longitudinal direction L can also have closed regions at both ends thereof, wherein the slit region B1 in this instance runs between these regions. Because a closed region in the collar region of the knee tends to impede an application of the bandage (and has to be pulled over the wound region), this embodiment is indeed intended to be within the scope of the invention but not as a preferred embodiment.

A ratio between the first compression strength of the first compression zone K1 and the second compression strength of the second compression zone K2 herein can be more than 1.5, or more than 2, or more than 3, or more than 3.5, or more than 4, or more than 4.5. The compression strength of the first compression zone K1 on a collar region, as in the knee region and ankle region shown in FIG. 1A, can be identical. However, said compression strength may also differ. The number of compression zones in the examples shown in FIGS. 1D, 1E, 2 and 4 is exemplary, and further compression zones with dissimilar compression strengths are possible. While it is therefore preferable that the compression strength of the first compression zone K1 is identical in the collar region on the knee as well is on the ankle, a third compression zone can be provided instead of the first compression zone K1 in the collar region on the ankle, the compression strength of said third compression zone being different from the compression strength in the collar region on the knee or deviating from the latter. In this case, however, the compression strength of this third compression zone is likewise greater than the compression strength of the second compression zone K2. The term compression strength is understood to mean a force or a pressure which acts radially inward on the body part enclosed by the bandage when the bandage is being applied, and which is caused by a circumferential elasticity or restoring force within the bandage (for example, as a result of incorporated elastane fibers or elastane threads in the circumferential direction transverse to the longitudinal direction L). In the field of compression stockings, compression classes CCL1 (moderate compression with a compression pressure of 2.4 kPA to 2.8 kPA), CCL2 (medium-strength compression with a compression pressure of 3.1 kPA to 4.3 kPA), CCL3 (strong compression with a compression pressure of 4.5 kPA to 6.1 kPA), and CCL 4 (extra-strong compression with a compression pressure of more than 6.5 kPA) are used for the compression strength in the context of the invention. In the field of compression stockings, compression classes CCL1 (moderate compression with a compression pressure of 2.4 kPA to 2.8 kPA), CCL2 (medium-strength compression with a compression pressure of 3.1 kPA to 4.3 kPA), CCL3 (strong compression with a compression pressure of 4.5 kPA to 6.1 kPA), and CCL 4 (extra-strong compression with a compression pressure of more than 6.5 kPA) are used for the compression strength in the context of the invention. The compression strength of the compression zones K1, K2 of the bandage 100 according to the invention here is preferably in the range of the compression class CCL1, or in the range of the compression class CCL2, or in the range of the compression class CCL3, or in the range of the compression class CCL 4, depending on the application and the intended purpose of the bandage 100. The term compression strength here always relates to a compression strength in an applied state of the bandage on a normally shaped lower leg or lower arm, which results from an elasticity or elongation stiffness in the circumferential direction of the closed bandage 100, corresponding to the compression strength, caused by elongation of the bandage material due to deflection. In this way, the term compression strength is to be considered equivalent to an elasticity modulus of the bandage material (or of the elastic support structure of elastomer threads) of a compression zone K1, K2 in the transverse direction QR or circumferential direction of the bandage 100.

In a further embodiment, the bandage 100 can also be combined with additional products for medical treatment, such as a foot bandage, for example. Shown in FIG. 1F is an example in which the bandage 100 is configured with a sock S or socklet. Such a sock is described in patent application DE 10 2016 104 206 A1, for example.

Schematic cutting patterns for producing the bandage 100 according to a plurality of exemplary embodiments are shown in FIG. 3. Illustrated on the left side is a cutting pattern for a calf region or a lower leg. The respective resultant cut edge portions A1, A2, A3 are indicated by arrows. However, the calf cutting pattern or lower leg cutting pattern is only exemplary. The profile of the cut edge portions A1, A2, A3 may vary, depending on the cutting pattern. Illustrated on the right side is a cutting pattern for a calf region or a lower leg, with a sock S. Shown here is a further cut edge portion A4 which is assigned to the sock S. The dashed line in both images furthermore indicates a resultant seam N which is created when two blanks Z of identical shape, or two of similar shape, obtained from the cutting pattern are connected to one another by ultrasonic welding.

The bandage part 10 herein can be produced from at least one blank Z of a flat material which comprises a non-woven material. A non-woven material which is provided for use in hospitals and care homes is preferably used here.

A non-woven material is a formation of fibers of finite length, continuous fibers (filaments) or cut yarns of any type and any origin, which are combined and connected to one another in any arbitrary way so as to form a non-woven fabric (a fibrous layer, a fibrous pile).

A non-woven material of synthetic and/or natural fibers can be provided as the non-woven material. Synthetic non-woven materials or mixtures of natural and synthetic non-woven materials have an increased elasticity. For ecological reasons, the non-woven material can comprise almost exclusively natural fibers in order to be ecologically degradable. For example, modal fibers or viscose fibers can be used as fibers. Furthermore, natural fibers have a better absorbency in comparison to synthetic fibers.

According to the invention, the non-woven material can be provided on a roll and/or bundle in such a manner that at least two cutting patterns with corresponding blanks can be processed in parallel. Such a blank Z is shown in FIG. 2 and FIG. 4. Corresponding cutting patterns are illustrated in FIG. 3. For example, the non-woven material can be supplied by the non-woven manufacturer on a roll, bundle or the like. By simultaneously processing a plurality of cutting patterns in parallel, the number of produced bandage parts 10 is significantly increased without creating increased costs.

The schematic cutting patterns illustrated in FIG. 3 have a calf shape or lower leg shape, as described above. However, the illustrated cutting patterns are only exemplary and further shapes can be provided, in particular with cut edge portions A1, A1′, A2, A2′, A3, A3′ of dissimilar profiles.

It is also possible that the bandage part 10 can be produced from at least one blank Z of a stretch material.

The stretch material herein can have an elasticity of at least 50% and a resilience of 30% to 100%.

The resilience describes to what extent a material returns to its original shape upon elongation. The term “elasticity” herein is intended to correspond to the term “stretch”. In this context, a material is interesting when it is elastic as well as partially resilient. Full resilience, thus a perfectly elastic behavior, is not necessary for the use of the bandage 100 as a single-use product. In this way, the material does not have to fully assume its original shape again upon elongation, but after initial contraction can remain in an elongated shape. In this way, the resilience can be less than 100%, wherein resilience of 100% corresponds to a perfectly elastic behavior. According to the invention, a resilience of 100% to 30%, or of 100% to 50%, or of 100% to 70%, is preferable. Elasticity corresponds to the ability to sustain an elongation without destruction such as tearing. The higher the elasticity, the more the material can be elongated in percentage.

The stretch material can have a 2-way stretch fabric, wherein one stretch direction extends in the transverse direction QR of the bandage 100, i.e. in only one direction QR. The transverse direction QR herein is perpendicular to the longitudinal direction L or runs perpendicularly to the cut edge portion A2, and in the applied state runs parallel to the cut edge portion of the upper and lower cuff region on the knee, or on the ankle.

The use of a 4-way stretch fabric is likewise possible, wherein one stretch direction extends in the transverse direction QR, and a further stretch direction extends in the longitudinal direction LR of the bandage 100. Depending on the requirements set for the bandage 100, the 2-way stretch fabric or the 4-way stretch fabric may be used. However, the use of a 2-way stretch material or a single-stretch material of which the stretch direction extends only in the transverse direction QR is more cost-effective to produce and has only minor disadvantages in comparison to a 4-way stretch material.

The blanks Z shown in FIG. 2, or the bandage parts 10 produced therefrom, respectively, are conceived for a leg region. Bandage parts 10 with dissimilar sizes are illustrated schematically herein. Shown in the lower image is the bandage part 10 including the sock S, which is obtained from the blank Z. The respective compression zones K1, K2 are highlighted by the shaded areas.

As has already been mentioned above, the stretch material can comprise elastomer threads or elastane threads of dissimilar thicknesses, which generate dissimilar compression strengths in the respective dissimilar compression zones K1, K2. Furthermore, the stretch material can comprise elastomer threads with dissimilar preloads, which generate dissimilar compression strengths in the respective dissimilar compression zones K1, K2.

Elastane fibers or elastane threads are synthetic filament yarns (yarn counts 11-2600 dtex) and are composed of at least 85% by weight of segmented polyurethane. They are distinguished in particular by a high elastic elongation. Under a tensile load, they can be elongated up to 6 to 8 times their original length, and once the tension has been released, immediately and completely assume their original length again. Elastane fibers can be readily dyed, are resistant to oxidation and can be produced so as to be more light-resistant and significantly thinner than rubber threads, so that finer and lighter woven fabrics and knitted fabrics can be generated therefrom than from rubber threads. Customary production methods for said elastane fibers include dry spinning (80% of world capacity), wet spinning, or reactive spinning. Said elastane fibers are resistant to sweat and cosmetic oils when appropriately cared for, can be readily laundered, have a moderate abrasion resistance, a density of 1.15 g/m, a moisture absorbance of 1.0-1.3%, a melting point of approximately 250° C., a softening point of 175° C., they turn yellow and degrade at more than 150° C. The maximum tensile force in terms of the initial yarn count is 4.5 cN/tex-12 cN/tex, the maximum tensile elongation is 420%-800%, the residual elongation after 300% elongation is 10%-30%, the bonding temperature is at more than 170° C. Brand names for elastomer threads or elastane threads include Lycra (Invista), Dorlastan (Asahi), creora (Hyosung) or Linel (Fillatice). While the use of elastane threads as elastomer threads is particularly preferable, other elastomer threads with comparable properties can of course also be used. The elasticity modulus of elastane threads here can be in a range between 0.01 GPa and 0.1 GPa. The elasticity modulus of the bandage material herein is to be understood to mean a local elasticity module in terms of the transverse direction QR, which is generated locally (thus within a region or a zone K1, K2 in the longitudinal direction L) in the carrier material as a result of elastomer threads which are incorporated in the carrier material such as a non-woven fabric, for example.

For example, the thread thickness in the first compression zone K1 can be in a range from 500 dtex to 1000 dtex, or in a range from 500 dtex to 900 dtex, or in a range from 500 dtex to 800 dtex, or in a range from 500 to 700 dtex, or in a range from 500 to 600 dtex, or in a range from 600 dtex to 800 dtex. In the second compression zone K2, the thread thickness can furthermore be in a range from 100 dtex to 200 dtex, or in a range from 100 dtex to 300 dtex, or in a range from 100 dtex to 400 dtex, or in a range from 100 dtex to 500 dtex, or in a range from 100 dtex to 600 dtex, or in a range from 300 dtex to 600 dtex. Furthermore, the thread spacing in the first compression zone can be in a range from 0.5 mm to 1 mm, or in a range from 0.1 mm to 10 mm, or in a range from 0.5 mm to 8 mm, or in a range from 1 mm to 7 mm, or in a range from 1 mm to 6 mm, or in a range from 1 mm to 5 mm, or in a range from 1 mm to 4 mm, or in a range from 1 mm to 3 mm, or in a range from 1 mm to 2 mm. In the second compression zone K2, said thread spacing can be in a range from 4 mm to 4.5 mm, or in a range from 3 mm to 5 mm, or in a range from 3 mm to 5.5 mm, or in a range from 2 mm to 6 mm, or in a range from 1 mm to 15 mm, or in a range from 2 mm to 12 mm, or in a range from 3 mm to 10 mm, or in a range from 4 mm to 8 mm, or in a range from 5 mm to 8 mm, or in a range from 6 mm to 8 mm. However, this is only exemplary and further thread thicknesses and spacings are possible. Furthermore, the threads can have a thread preload of more than 50%, or more than 100%, or more than 150%, or more than 200%, or more than 250%, or more than 300%, or more than 350%, or more than 400%, or more than 450%, or more than 500%, and/or less than 600%.

As can be seen from FIG. 1A, the bandage part 10 next to the slit region B1 can comprise the distal region B2 which is closed in a tubular manner and when applying the bandage 100 is pulled over a foot or a hand of the living being, and in the applied state of the bandage 100 is adjacent to the foot or the hand. The distal region B2 is likewise indicated in a simplified manner by a corresponding arrow in FIG. 1A.

The bandage 100 can thus be easily and comfortably applied by means of the closed region B2, and be closed by the corresponding fastening elements 22, 22′. The closed region is first positioned at a corresponding location (not a wound location), for example on an ankle or on a wrist, the loose slit part, or the loose flap of the slit region B1 of the bandage part 10, can then be easily closed in a releasable manner by means of the fastening elements 22, 22′. The application of the bandage 100 is further simplified as a result.

The bandage part 10 herein, in an applied state, in its distal region B2 which is closed in a tubular manner can enclose in a compressing manner an ankle region of a foot, and in its slit region B1 can enclose in a compressing manner a calf region of a lower leg. For example, the bandage 100 herein can function as a biocompatible physical bandage layer which protects and supports the skin of a living being.

As has already been described above, the fastening part 20 as fastening elements 22, 22′ can comprise individual hook and loop fastener parts which are punched out of a hook and loop fastener tape and are individually connected to the bandage part 10.

In a further embodiment, the fastener part 20 can also be configured as an integral multi-hook and loop fastener part which on the one side thereof, in its non-hook region, is connected to the bandage part 10, and on the opposite side thereof, in its hook region, has projecting fastener elements 22, 22′ which are configured by punching the hook and loop fastener tape.

The releasable fastening elements 22, 22′ make it possible for the bandage 100 to be opened in an arbitrary manner, for example for checking a healing process of a wound, or easily replacing a corresponding wound dressing such as a wound plaster or a pad. No unnecessary winding as in conventional bandages is required here.

The fastener elements 22, 22′ described above, instead of or in addition to the hook and loop fastener elements described above, can have hook fastener elements, or adhesive tape fastener elements (for example with a tear-off backing film).

Such a multi-hook and loop fastener part is illustrated in FIG. 4, for example, wherein the punching of the fastener elements 22 is illustrated in a simplified manner. Shown in FIG. 4 are two blanks Z which are disposed on top of one another and are removed from a non-woven material or stretch material and are subsequently further processed in stages.

According to a method for producing the bandage 100, the non-woven material, or the stretch material, can be provided on a roll and/or a bundle, as has already been described above, wherein two pieces can be removed from the non-woven material or the stretch material according to a predefined cutting pattern, such as has been described with reference to FIG. 3. The removal of the pieces from the non-woven material or the stretch material can take place by cutting or punching. The blanks Z of identical shape obtained herein can be simultaneously connected to one another at corresponding end regions by ultrasonic welding. For example, the blanks Z can be connected to one another by means of a seam which is elastic in a seam direction N. The seam direction N is illustrated as a dashed line in FIGS. 1A, 1B, 1F and FIG. 3.

The blanks Z can subsequently be further processed, wherein in a following processing step non-woven material or stretch material is omitted on the end regions. For example, on an upper side of the blank Z illustrated in FIG. 4, a material which is assigned to the fastener part 20 can be connected to the blank Z instead of the omitted material. The fastener elements 22 can then be punched out of said material assigned to the fastener part 20, so as to form the bandage part 10.

In a further embodiment, a blank Z can also be removed and processed integrally. The fastener part 20 herein can be attached to a lateral end, for example. The bandage part 100 can then by formed by folding the blank Z. The slit region B1 here extends along the entire longitudinal direction LR. A resultant fold line extends in the longitudinal direction LR on the side opposite the fastener part 20. In this case however, this side is not curved and adapted to a calf shape, but is ideally straight in order to guarantee easy folding.

As has been described with reference to FIG. 3, the bandage part 10 can have a shape adapted to the calf region of a lower leg. As a result the bandage 100 can provide an optimal fit from the outset. Further shapes are possible. Likewise, the socklet or the sock S of the further embodiment can also provide a corresponding shape adapted to a foot.

As already described, the bandage 100 can be configured as a hygienic single-use product.

The bandage 100 according to the invention provides an adaptive ready-to-use lower leg bandage (for single use), which covers the lower leg from the ankle to below the knee. It is a purpose of the bandage 100 according to the invention to act as a biocompatible physical layer so as to protect and support the skin. The bandage 100 is intended to be used on its own or in conjunction with other products for medical treatment. When applied to injured skin, said bandage 100 serves as a secondary or tertiary bandage layer about the lower leg. The bandage 100 has padding properties, can contribute toward protecting underlying layers (e.g. wound dressings), and can absorb excess liquid to a limited extent. The bandage 100 is a non-sterile, non-active, non-invasive product.

The bandage 100 according to the invention is furthermore suitable for application under compression, and can be readily combined with a ready-to-use foot bandage.

For example, the bandage 100 can be used with patients that receive short-term or long-term medical treatment of the lower leg, or are bed-ridden or have restricted mobility.

An exemplary application of the bandage 100 according to the invention for the right or left lower leg will be explained hereunder.

First, the bandage 100 is selected by means of a size table. The following points are to be observed in the selection. The thickness of a wound dressing has to be taken into account. Likewise, variations in the circumference of the lower leg in the course of the day due to edema formation and retraction are possible. The use of another product size may become necessary in the healing process or due to a change in the type of care. A proper treatment of existing wounds must take place prior to application. The bandage 100 should be carefully pre-stretched at the lower collar (and be held at the seam locations so as to avoid tearing in the process). The bandage 100 has to be applied in such a way that the opening with hook and loop fasteners points toward the front. The lower collar is then carefully pushed over the foot and pulled up to the ankle. In the process, the tabs of the opening are to be gripped close to the end of the seam. Optionally, the collar can be expanded from the inside by hand, in order to reduce friction on the foot. The fasteners are to be fixed in a preliminary manner in order, working from the bottom to the top. Care has to be taken that the two sides of the opening overlap sufficiently. The fasteners are subsequently readjusted. The bandage 100 should bear smoothly and not have any creases. After use, the bandage is pulled off and disposed of. Furthermore, the hook and loop fasteners are to be readjusted (loosened/widened) if (a) there is a sense of constriction, (b) the bandage 100 no longer has elasticity at any location, or is highly tensioned, (c) the bandage 100 slips. Application of the bandage 100 as primary layer: on skin which is intact and without injuries. Application of the bandage 100 as secondary layer: on injured skin or open wounds with an underlying primary layer (wound dressing).

1. A bandage (100) for enclosing a leg region or arm region of a living being, having:
an elongate, tubular bandage part (10) which is at least partially slit once in the longitudinal direction (LR) and which in an applied state is adapted to elastically enclose the leg region or arm region; and
a fastener part (20) which has fastener elements (22) and is fixedly connected to one end of the slit region (B) of the bandage part (10) so as to releasably close the slit region (B) by way of an overlapping connection of the adjacent ends of the slit region (B) by means of the fastener elements (22, 22′), and wherein the bandage part (10) in the longitudinal direction (LR) has dissimilar compression zones (K1, K2) having in each case dissimilar radial compression strengths transverse to the longitudinal direction (L).

2. The bandage (100) of claim 1, wherein the dissimilar compression zones (K1, K2) are assigned to predetermined regions of a leg.

3. The bandage (100) of claim 2, wherein a first compression zone (K1) in the bandage part (10) is assigned to a collar region on the knee and/or on the ankle of the lower leg, and in that a second compression zone (K2) in the bandage part (10) is assigned to a calf region of the lower leg, wherein the first compression zone (K1) has a higher compression strength than the second compression zone (K2).

4. The bandage (100) of claim 1, wherein the bandage part (10) is produced from at least one blank (Z) of a flat material which comprises a non-woven material and/or is configured as a hygienic single-use product.

5. The bandage (100) of claim 1, wherein the bandage part (10) is produced from at least one blank (Z) of a stretch material.

6. The bandage (100) of claim 5, wherein the stretch material has an elasticity of at least 50% and a resilience of 30% to 100%.

7. The bandage (100) of claim 5, wherein the stretch material has a 2-way stretch fabric, wherein one stretch direction extends in the transverse direction (QR) of the bandage (100).

8. The bandage (100) of claim 5, wherein the stretch material has a 4-way stretch fabric, wherein one stretch direction extends in the transverse direction (QR) and a further stretch direction extends in the longitudinal direction (LR) of the bandage (100).

9. The bandage (100) of claim 5 wherein the stretch material comprises elastomer threads of dissimilar thicknesses which generate dissimilar compression strengths in the respective dissimilar compression zones (K1, K2).

10. The bandage (100) of claim 5, wherein the stretch material comprises elastomer threads with dissimilar preloads which generate dissimilar compression strengths in the respective dissimilar compression zones (K1, K2).

11. The bandage (100) of claim 1, wherein the bandage part (10) next to the slit region (B1) comprises a distal region (B2) which is closed in a tubular manner and when applying the bandage (100) is pulled over a foot or a hand of the living being, and in the applied state of the bandage (100) is adjacent to the foot or the hand.

12. The bandage (100) of claim 11, wherein the bandage part (10) in an applied state, in its distal region (B2) which is closed in a tubular manner, encloses in a compressing manner an ankle region of a foot, and in its slit region (B1) encloses in a compressing manner a calf region of a lower leg and/or has a shape adapted to the calf region of a lower leg.

13. The bandage (100) of claim 1, wherein the fastener part (20) as fastener elements (22, 22′) comprises individual hook and loop fastener parts which are punched out of a hook and loop fastener tape and are individually connected to the bandage part (10).

14. The bandage (100) of claim 1, wherein the fastener part (20) is configured as an integral multi-hook and loop fastener part which on the one side thereof, in its non-hook region, is connected to the bandage part (10), and on the opposite side thereof, in its hook region, has projecting fastener elements (20, 22′) which are configured by punching the hook and loop fastener tape.

15. The bandage (100) of claim 1, wherein the bandage part (10) is produced from two blanks (Z) of identical shape, which are connected to one another by a seam which is elastic in a seam direction (N).
